(19) United States
(12) Patent Application Publication
(10) Pub. No.: US 2006/0276586 A1
(43) Pub. Date: Dec. 7, 2006

(54) Peg-physiologically active polypeptide homodimer complex having prolonged in vivo half-life and process for the preparation thereof

(75) Inventors: Young Min Kim, Kyungki-do (KR); Dae Jin Kim, Seoul (KR); Sung Min Bae, Seoul (KR); Chang Ki Lim, Seongnam-si (KR); Kyeong Bae Kim, Seoul (KR); Se Chang Kwon, Seoul (KR); Gwan Sun Lee, Seoul (KR)

Correspondence Address: SUGHRUE MION, PLLC, 2100 PENNSYLVANIA AVENUE, N.W., SUITE 800, WASHINGTON, DC, 20037, US

(73) Assignee: HANMI PHARM. CO. LTD, Kyungki-do (KR)

(21) Appl. No.: 10/551,764

(22) PCT Filed: Apr. 3, 2004
(86) PCT No.: PCT/KR04/00781

§ 371 Date: Oct. 3, 2005

(30) Foreign Application Priority Data
Apr. 3, 2003 (KR) 10-2003-0021122

(51) Int. Cl. C08G 63/91; C07K 14/53; C07K 14/56

(52) U.S. Cl. 525/54.1; 530/351; 530/399

(57) ABSTRACT

A PEG-polypeptide homodimer complex, which comprises a PEG linker and two molecules of a physiologically active polypeptide, wherein the two molecules of the physiologically active polypeptide are connected via the PEG linker, and each of the two molecules of the physiologically active polypeptide is modified with one molecule of PEG, is useful for the development of a polypeptide drug having a prolonged half-life in the blood.

### FIELD OF THE INVENTION

The present invention relates to a PEG-physiologically active polypeptide homodimer complex having a prolonged in vivo half-life and a process for the preparation thereof.

### BACKGROUND OF THE INVENTION

Polypeptides are susceptible to denaturation or enzymatic degradation in the blood, liver or kidney. Because of the low stability of polypeptides, it has been required to administer polypeptide drugs at a predetermined frequency to a subject in order to maintain an effective plasma concentration of the active substance. Moreover, since polypeptide drugs are usually administered by infusion, frequent injection thereof causes considerable discomfort to a subject. Thus, there have been many studies to develop a polypeptide drug which has an increased circulating half-life in the blood, while maintaining a high pharmacological efficacy. Such a polypeptide drug should also meet the requirements of enhanced serum stability, high activity, applicability to various polypeptides and a low probability of inducing an undesirable immune response when injected into a subject.

One of the most widely used methods for improving the stability of a polypeptide is the chemical modification thereof with a highly soluble macromolecule such as polyethylene glycol (“PEG”) which prevents the polypeptide from contacting with proteases. It is also well known that, when linked to a polypeptide drug specifically or non-specifically, PEG increases the solubility of the polypeptide drug and prevents the hydrolysis thereof, thereby increasing the serum stability of the polypeptide drug without incurring any immune response due to its low antigenicity (Sada et al., J. Fermentation Bioengineering, 1991, 71: 137-139). However, such pegylated polypeptide tends to have low activity as the molecular weight of PEG increases, because PEG randomly forms a covalent bond with the free lysine residue of the polypeptides.

Methods of selectively pegylating a specific site of a polypeptide to maintain the activity of the polypeptide are disclosed in U.S. Pat. Nos. 5,766,897 and 5,985,265. However, they do not show any distinctive merits in terms of prolonged activity of the polypeptides in vivo.

Accordingly, there has continued to exist a need to develop a polypeptide complex having a satisfactory activity and prolonged in vivo half-life.

### SUMMARY OF THE INVENTION

It is, therefore, an object of the present invention to provide a PEG-physiologically active polypeptide homodimer complex prepared by making a homodimer by connecting specific parts of two molecules of a physiologically active polypeptide by a PEG linker having a small molecular weight, and modifying the homodimer with a PEG having a large molecular weight, thereby minimizing the decrease of the biological activity thereof, and increasing the physiologically active polypeptide in vivo stability to prolong the peptide's in vivo activity.

It is another object of the present invention to provide a method for preparing the PEG-physiologically active polypeptide homodimer complex.

### BRIEF DESCRIPTION OF THE DRAWINGS

The above and other objects and features of the present invention will become apparent from the following description of the invention, when taken in conjunction with the accompanying drawings, in which:

FIG. 1 is a SDS-PAGE gel photograph of a hGH homodimer and a di-PEG-hGH homodimer complex in accordance with the present invention;

FIG. 2A shows a pharmacokinetic graph comparing the in-blood half-life of a mono-PEG-hGH with that of a di-PEG-hGH homodimer complex in accordance with the present invention;

FIG. 2B presents a pharmacokinetic graph comparing the in-blood half-life of a mono-PEG-IFN with that of a di-PEG-IFN homodimer complex in accordance with the present invention;

FIG. 2C offers a pharmacokinetic graph comparing the in-blood half-life of a mono-PEG-G-CSF with that of a di-PEG-G-CSF homodimer complex in accordance with the present invention; and

FIG. 3 depicts a diagram showing the result of a weight increase test conducted with pituitary-removed rats, which compares the in vivo activity of a mono-PEG-hGH with that of a di-PEG-hGH homodimer complex in accordance with the present invention.

### DETAILED DESCRIPTION OF THE INVENTION

In accordance with one aspect of the present invention, there is provided a PEG-polypeptide homodimer complex comprising a PEG linker and two molecules of a physiologically active polypeptide, wherein the two molecules of the physiologically active polypeptide are connected via the PEG linker, and each of the two molecules of the physiologically active polypeptide is modified with one molecule of PEG.

Physiologically active polypeptides which may be employed in a preferred embodiment of the invention include human growth hormnone (hGH), interferon (IFN), granulocyte colony-stimulating factor (G-CSF), granulocyte colony-stimulating factor derivative having an amino acid sequence wherein the 17^{th} cysteine is substituted with serine (¹⁷S-G-CSF), erythropoietin (EPO), insulin, interleukin, granulocyte macrophage colony-stimulating factor (GM-CSF) and tumor necrosis factor receptor (TNFR). The physiologically active polypeptides, to which the present invention can be applied, are not limited to those recited above; but may include any physiologically active polypeptides useful for prolonging in vivo half-life.

The physiologically active polypeptide of the present invention may be either in a native form isolated from a mammal or chemically synthesized. Further, the pglypeptide may also be prepared from a transformed prokaryotic or eukaryotic cell by genetic engineering.

In a preferred embodiment of the invention, the PEG linker may be hydrophilic so that the homodimer does not precipitate in an aqueous medium. Further, the PEG linker may have reactive groups at both ends so as to combine specifically with each amino terminal group of the two molecules of the physiologically active polypeptide. The suitable reactive group of the PEG linker may be an aldehyde or propionic aldehyde group.

In a preferred embodiment of the invention, the molecular weight of the PEG linker may range from 1 to 100 kDa, more preferably 2 to 20 kDa.

In a preferred embodiment of the invention, the PEG molecule may be a customary water-soluble PEG molecule, which may combine with the ε-amino group of a lysine, cysteine or histidine residue of a polypeptide depending on the active group of the PEG.

In a preferred embodiment of the invention, the molecular weight of the PEG which is used to modify the two molecules of the physiologically active polypeptide may range from 1 to 100 kDa, more preferably 20 to 40 kDa.

It is preferable that the reactive group of the PEG molecule is a maleimide or succinamide group; and the succinamide derivative may include succinimidyl propionate, succinimidyl carboxymethyl and succinimidyl carbonate.

Further, the PEG molecule used in the present invention may be linear or branched, while a branched one is preferred.

In accordance with another aspect of the present invention, there is provided a method for preparing the PEG-polypeptide homodimer complex, which comprises the steps of:
(a) preparing a homodimer by connecting two molecules of a physiologically active polypeptide via a PEG linker; and
(b) modifying each physiologically active polypeptide of the homodimer with one molecule of PEG.

In accordance with a preferred embodiment of the present invention, the molar ratio of the physiologically active polypeptide to the PEG linker used in step (a) is preferably in the range of 1:0.25 to 1:10, more preferably from 1:0.5 to 1:1.

In a preferred embodiment of the invention, step (a) may be performed at a temperature ranging from 2 to 10° C. in the presence of a reducing agent which may be selected from the group consisting of sodium cyanoborohydride, sodium borohydride, dimethylamine borate, trimethylamine borate and pyridine borate.

After the completion of step (a), the polypeptide homodimer so formed may be isolated utilizing any of the conventional methods useful for purifying proteins, such as size exclusion chromatography and ion exchange chromatography.

After completion of PEG modification of the polypeptide homodimer in step (b), the homodimer complex so formed may be obtained using size exclusion chromatography.

The following Examples are intended to further illustrate the present invention without limiting its scope.

### EXAMPLE 1

### Preparation and Purification of hGH Homodimer

A recombinant hGH was prepared in accordance with the method of Korean Patent No. 316,347, and the hGH of the present invention was a native form. 5 mg/ml of hGH solution was prepared by dissolving the hGH prepared above in 100 mM phosphate buffer. A PEG linker having aldehyde groups at both ends and a molecular weight of 3.4 kDa (Shearwater Inc., USA) was added to the hGH solution in an amount corresponding to hGH:PEG linker molar ratio of 1:0.5, 1:1, 1:2.5, 1:5, 1:10, or 1:20 to connect the hGH and the PEG linker. A reducing agent, sodium cyanoborohydride (NaCNBH₃), was then added to a final concentration of 20 mM. The reaction mixture was stirred at 4° C. for 3 hours, and was subjected to size exclusion chromatography using Superdex 200 (Pharmacia) to separate the hGH homodimer (hGH-PEG linker-hGH) which has the PEG linker selectively connected to each of the amino terminals of the two hGH molecules. The hGH homodimer was eluted using 50 mM sodium phosphate buffer (pH 8.0), and unreacted hGH and PEG linker were removed. It was found that the optimum hGH:PEG linker molar ratio for obtaining the homodimer was in the range from 1:0.5 to 1:2. The hGH homodimer fraction obtained above was further purified by an anion exchange resin column. Specifically, 3 ml of PolyWAX LP column (Polywax Inc., USA) was equilibrated with 10 mM Tris-HCl buffer solution (pH 7.5), the hGH homodimer fraction was loaded onto the column at a rate of 1 ml/minute, and the column was washed with 5 column volume (15 ml) of the Tris-HCI buffer solution. The hGH homodimer was separated from mono PEG linker coupled with one hGH molecule by a salt concentration gradient method, applying 10 column volume (30 ml) of 1 M NaCl buffer over 30 minutes at a varying concentration gradient in the range of 0 to 100%.

### EXAMPLE 2

### Preparation of hGH Homodimer Modified with Branched 40 kDa PEG

A branched N-hydroxysuccinimidyl-PEG (NHS-PEG) having a molecular weight of 40 kDa (Shearwater Inc., USA) was allowed to react with the lysine residue of the hGH homodimer obtained in Example 1 in 100 mM sodium phosphate buffer (pH 8.0) at room temperature for 2 hours. The homodimer: NHS-PEG molar ratio was varied among 1:2, 1:5, 1:10, and 1:20. A size exclusion chromatography using Superdex was performed upon completion of the reaction to purify di-PEG-hGH homodimer, each of the two hGH molecules thereof being modified with one molecule of NHS-PEG. Phosphate buffered saline was used as a buffer solution to remove unmodified hGH homodimer and mono-NHS-PEG-hGH homodimer having only one molecule of NHS-PEG connected thereto. The ratio of the mono-NHS-PEG-hGH homnodimer and di-PEG-hGH homodimer products was about 60%:40%. It was found that the optimal hGH homodimer to NHS-PEG molar ratio for obtaining the di-PEG-hGH homodimer was 1:10.

### EXAMPLE 3

### Preparation of IFN Homodimer Modified with Branched 40 kDa PEG

An IFN homodimer (IFN-PEG linker-IFN) was prepared in accordance with Example 1, and the IFN homodimer was modified with two molecules of branched NHS-PEG having a molecular weight of 40 kDa as in Example 2, employing IFN instead of hGH. The ratio of the mono-PEG-IFN homodimer and di-PEG-IFN homodimer products was about 60%:40%.

### EXAMPLE 4

### Preparation of G-CSF Homodimer Modified with Branched 40 kDa PEG

A G-CSF homodimer (G-CSF-PEG linker-G-CSF) was prepared in accordance with Example 1, and the G-CSF homodimer was modified with two molecules of branched NHS-PEG having a molecular weight of 40 kDa as in Example 2, using G-CSF instead of hGH. The ratio of the mono-PEG-G-CSF homodimer and di-PEG-G-CSF homodimer products was about 60%:40%.

### COMPARATIVE EXAMPLE 1

### Preparation of hGH Monomer Modified with Branched PEG

Three hGH solutions of 1 mg/ml were prepared by dissolving the hGH in 100 mM phosphate buffer solution, and then, a branched methoxy-PEG-aldehyde (Shearwater Inc, USA) having a molecular weight of 40 kDa was added thereto in an amount corresponding to an hGH:PEG molar ratio of 1:4. Sodium cyanoborohydride (NaCNBH₃, Sigma) was added thereto to a final concentration of 20 mM, and the reduction mixture was gently stirred at 4° C. for 18 hrs. To separate the mono-PEG-hGH having an PEG molecule linked to an amino-terminal group of hGH, the reaction mixture was subjected to anion exchange chromatography. The pegylated reaction mixture was loaded onto a PolyWAX LP column (Polywax Inc., USA) equilibrated with 10 mM Tris-HCl buffer (pH 7.5), eluted at a rate of 1 ml/minute, and the column was washed with 5 column volume (15 ml) of the same buffer. And then, the tri-, di- and monoPEG-hGH fractions were separated from the resultant by a salt concentration gradient method, applying 10 column volume (30 ml) of 1 M NaCl buffer solution over 30 minute automatically changing the concentration gradient from 0 to 100%.

The mono-PEG-hGH fraction was concentrated, loaded onto a Superdex 200 (Pharmacia, USA) size exclusion chromatography equilibrated with 10 mM sodium phosphate buffer (pH 7.0) and eluted with the same buffer at a flow rate of 1 ml/minute. The tri- and di-PEG-hGH which eluted earlier than the mono-PEG-hGH were removed, to obtain purified mono-PEG-hGH.

### COMPARATIVE EXAMPLES 2 AND 3

### Preparation of IFN Monomer and G-CSF Monomer Modified with Branched PEG, Respectively

An IFN monomer modified with a branched PEG and a G-CSF monomer modified with a branched PEG were each prepared and purified according to the same method described in Comparative Example 1, being IFN (Comparative Example 2) and G-CSF (Comparative Example 3), respectively, instead of hGH.

### TEST EXAMPLE 1

### Confirmation and Quantification of PEG Complex

Polypeptide complexs prepared in the above Examples were each analyzed for its concentration and purity by Coomassie dyeing, SDS-PAGE and size exclusion chromatography (HPLC), and the concentration was detected at 280 nm in accordance with the Beer-Lambert law (Bollag et al., Protein Methods Chapter 3, press in Wiley-Liss).

The apparent molecular weight of hGH homodimer was about 48 kDa, and those of the IFN homodimer and G-CSF homodiner were similar. When modified with one molecule of PEG having a molecular weight of 40 kDa, the apparent molecular weight of the mono-PEG-hGH homodimer was about 150 kDa; and when modified with two molecules of 40 kDa PEG, the molecular weight of the di-PEG-hGH homodimer complex was 240 kDa. Meanwhile, the molecular weight of mono-PEG-hGH was about 120 kDa, and those of IFN and G-CSF were similar.

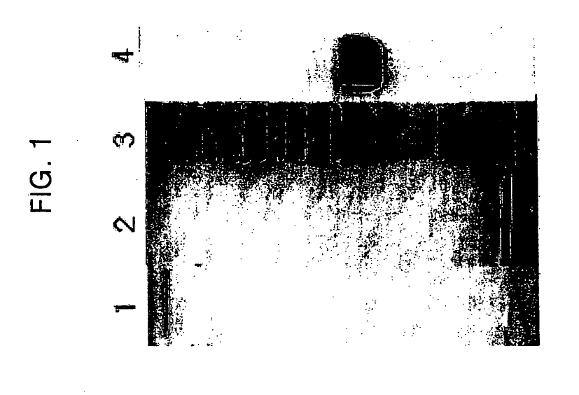

FIG. 1 shows the SDS-PAGE results obtained for the hGH (rail 1), hGH homodimer (rail 2), and di-PEG-hGH homodimer complex (rail 4), respectively.

Rail 3 is a standard molecular weight protein (Invitron, bench marker which means 40, 50, 60, 70, 80, 90, 100, 120, 160 and 220 kDa from the bottom). As shown in FIG. 1, the apparent molecular weight of di-PEG-hGH homodimer complex is about 240 kDa and the complex is highly pure in view of the appearance of a single band.

### TEST EXAMPLE 2

### Measurement of In Vitro Activity of di-PEG-hGH Homodimer Complex

In vitro activities of the di-PEG-hGH homodimer complex (Example 2) and the mono-PEG-hGH (Comparative Example 1) were measured using rat node lymphoma cell line Nb2 (European Collection of Cell Cultures, ECCC #97041101) which undergo hGH dependent mitosis, as follows.

Nb2 cells were cultivated in Fisher's medium supplemented with 10% fetal bovine serum (FBS), 0.075% NaCO₃, 0.05 mM 2-mercaptoethanol and 2 mM glutamine. The cells were incubated for additional 24 hours in the same medium without 10% FBS. After about 2×10⁴ cells per well were added to a 96-well plate, various dilutions of di-PEG-hGH homodimer complex and mono-PEG-hGH, wild-type hGH and a control (National Institute for Biological Standards and Control, NIBSC) were added to each well and the plate was incubated for 48 hours at 37° C. in a CO₂ incubator. To measure the extent of cell growth (the number of cells existed in each well), 25 μl of cell titer 96 Aqueous One Solution (Promega, USA) was added to each well and incubated for 4 hours.

Absorbance at 490 nm was measured to calculate the titer of each sample, and the calculated titers are shown in Table 1.

TABLE 1
In vitro activity analysis of hGH

| | | | Relative activity |
|---|---|---|---|
| | Conc. (ng/ml) | In vitro activity | (%) |
| Wild-Type hGH | 100 | 5.85E+06 | 100 |
| Control (NIBSC) | 100 | 5.02E+06 | 88.9 |
| Mono-PEG-hGH | 100 | 4.65E+05 | 7.8 |
| (Comp. Ex. 1) | | | |
| Di-PEG-hGH | 100 | 2.17E+04 | 0.8 |
| homodimer complex | | | |
| (Ex. 2) | | | |

As can be seen from Table 1, the in vitro activity of PEG modified hGH was lower than that of the unmodified hGH.

### TEST EXAMPLE 3

### Measurement of In Vitro Activity of di-PEG-IFN Homodimer Complex

In vitro activities of the di-PEG-IFN homodimer complex (Example 3) and the mono-PEG-IFN (Comparative Example 2) were measured by a cell culture biopsy method using Madin-Darby bovine kidney cells (MDBK cells; ATCC CCL-22) saturated with vesicular stomatitis virus (VSV). IFN α 2b having no PEG modification (NIBSC IFN) was employed as a control.

MDBK cells were cultured in MEM (minimum essential medium, JBI) supplemented with 10% FBS and 1% penicillin-streptomycin at 37° C. in a 5% CO₂ incubator. Samples and a control (NIBSC IFN) were diluted with the same culture medium to a constant concentration, and 100 μl of each dilution was distributed to a 96-well plate. 100 μl of the cultured cell solution was added to each well, and the cells were incubated at 37° C. for about 1 hr in a 5% CO₂ incubator. After an hour, 50 μl pt of VSV having a viral concentration of 5 to 7×10³ PFU was added to each well, and further incubated for 16 to 20 hours at 37° C. under 5% CO₂. Wells containing only cells and virus without samples or the control were employed as a negative control, and wells containing only cells without added viruses, as a positive control.

To remove the culture medium and to stain living cells, 100 μl of a neutral red solution was added to each well and further incubated at 37° C. for 2 hours in a 5% CO₂ incubator. After removing the supernatant by aspirating, the extraction solution (100 μl of a mixture of 100% ethanol and 1% acetate (1:1)) was added to each well. The stained cells were resuspended in the extraction solution with shaking and the absorbance at 540 nm was measured. ED₅₀ representing 50% of the maximum cell growth was calculated based a regarding the cell growth of the positive control as 100% relative to the cell growth of the negative control.

TABLE 2
In vitro activity analysis of IFN α

| | | | Relative activity |
|---|---|---|---|
| | Conc. (ng/ml) | ED₅₀ (IU/mg) | (%) |
| Wild-type IFN α | 100 | 4.24E+08 | 100 |
| Mono-PEG-IFN | 100 | 1.02E+07 | 2.4 |
| (Comp. Ex. 2) | | | |
| Di-PEG-IFN | 100 | 1.20E+05 | 0.03 |
| homodimer complex | | | |
| (Ex. 3) | | | |

As shown in Table 2, the in vitro activity of PEG modified IFN was lower than that of the unmodified IFN.

### TEST EXAMPLE 4

### Measurement of In Vitro Activity of di-PEG-G-CSF Homodimer Complex

In vitro activities of the di-PEG-G-CSF homodimer complex (Example 4) and the mono-PEG-G-CSF (Comparative Example 3) were measured, as follows.

First, human myelogenous originated cells,. HL-60 (ATCC CCL-240, Promyelocytic leukemia patient/36 yr old Caucasian female) cells, were cultivated in RPMI 1640 medium supplemented with 10% FBS, and the number of cells were adjusted to about 2.2×10⁵ cells/ml. DMSO (dimethylsulfoxide, culture grade/SIGMA) was added to the cells to a concentration of 1.25% (v/v). 90 μl of the DMSO treated culture solution having about 2×10⁴ suspended cells per well was added to 96-well plate (Coming/low evaporation 96 well plate) and incubated at 37° C. for 48 hours in a 5% CO₂ incubator.

Samples and a control (NIBSC G-CSF) were diluted with RPMI 1640 medium at a proper ratio to a concentration of 500 ng/ml, and the resulting solutions were subjected to 10 cycles of sequential half dilution with the same medium. 10 μl se of each sample prepared above was added to each well having HL-60 cells on cultivation, and the concentration was reduced by half from 50 ng/ml. The microplates treated with samples were further incubated at 37° C. for 48 hour.

To examine the extent of cell growth after the incubation, the number of cells were determined by measuring absorbance at 670 nm using CellTiter96™ (Promega, USA).

TABLE 3
In vitro activity analysis of G-CSF

| | | Relative activity |
|---|---|---|
| | ED₅₀ (ng/ml) | (%) |
| Wild-type G-CSF | 0.30 | 100 |
| Mono-PEG-G-CSF | 9.7 | 3.1 |
| (Comp. Ex. 3) | | |
| Di-PEG-G-CSF | 2.5 | 12 |
| homodimer complex | | |
| (Ex. 4) | | |

As can be seen from Table 3, the in vitro activity of PEG modified G-CSF was lower than that of the unmodified G-CSF. However, the activity relative to wild-type G-CSF of the di-PEG-G-CSF homodimer complex of the present invention (%) was about 4-fold higher than that of mono-PEG-G-CSF, unlike those of hGH and IFN. These results show that the inventive di-PEG-G-CSF homodimer complex exhibits high in vitro activity due to the formation of G-CSF homodimer.

### TEST EXAMPLE 5

### Pharmacokinetics Analysis

5 Sprague-Dawley (SD) rats were used for each group in the following experiments. Mice received subcutaneous injections of 100 μg/kg of a biologically active wild-type protein (control group), and polypeptide complexs (test group) prepared in Examples and Comparative Examples, respectively. Blood samples were taken from the control group at 0.5, 1, 2, 4, 6, 12, 24, 30 and 48 hour after the injection, and the samples of the test groups, at 1, 6, 12, 24, 30, 48, 72, 96 and 120 hours after the injection. Blood samples were collected in a tube coated with heparin to prevent blood coagulation, and subjected to high-speed micro centrifugation at 4° C., 3,000×g for 5 minute to remove cells. The protein concentration in sera was measured by ELISA method using the respective antibody specific for each biologically active polypeptide.

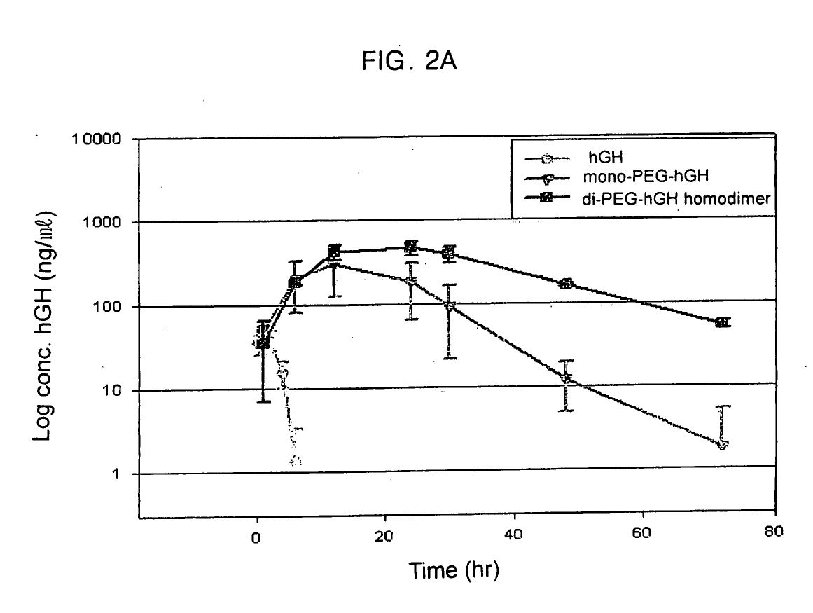

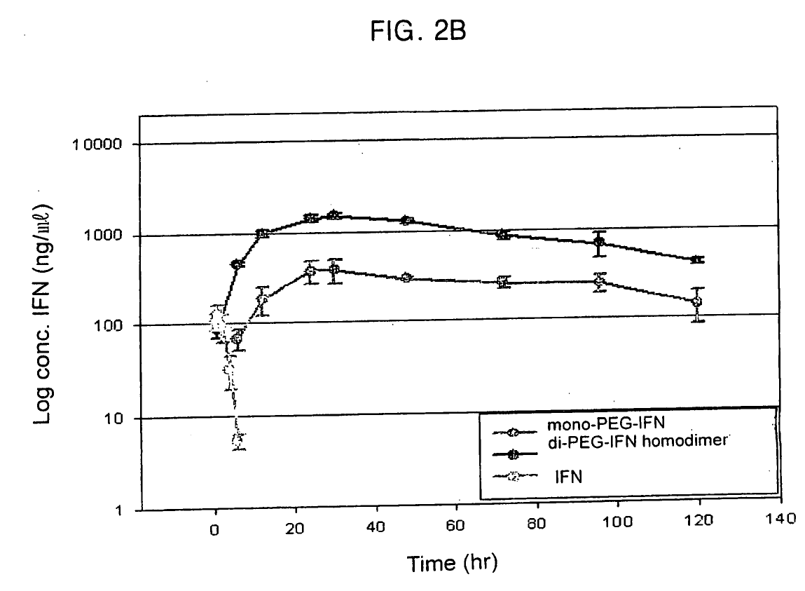

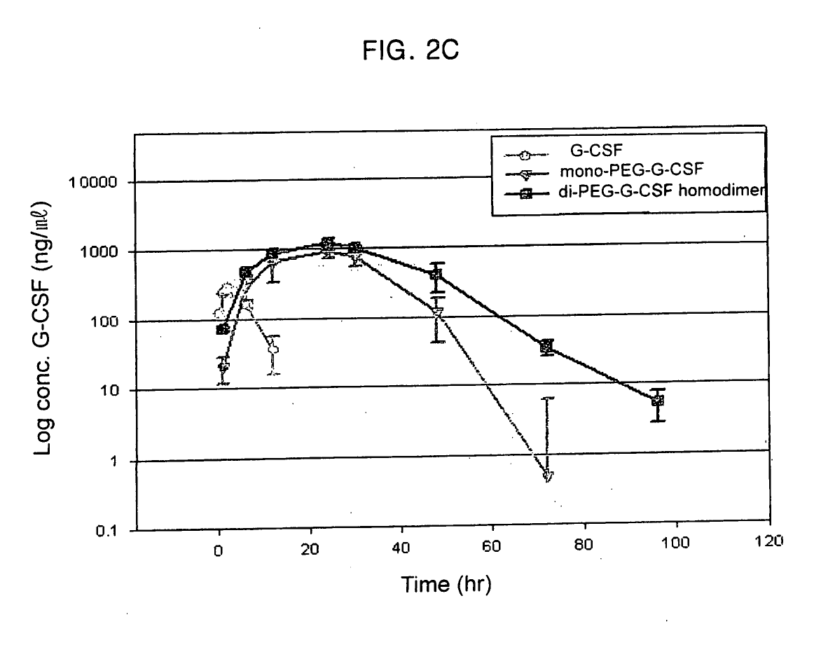

Pharmacokinetic graphs of the wild-type protein and polypeptide complexes are shown in FIGS. 2A to 2C, respectively, and T_{1/2} (half-life of a drug in blood), in Table 4.

TABLE 4
T_{1/2} of each wild-type protein and polypeptide complex (hr)

| Protein | hGH | IFN | G-CSF |
|---|---|---|---|
| Wild-type protein | 1.1 | 1.7 | 2.8 |
| Mono-PEG- | 7.7 | 49.3 | 4.3 |
| polypeptide complex | (Comp. Ex. 1) | (Comp. Ex. 2) | (Comp. Ex. 3) |
| Di-PEG-polypeptide | 15.8 | 73.8 | 8.9 |
| homodimer complex | (Ex.2) | (Ex. 3) | (Ex. 4) |

As can be seen in Table 4, the half-life of each of the di-PEG-polypeptide homodimer complexes was much higher than that of wild-type protein and about 2-fold higher than that of the corresponding mono-PEG-polypeptide prepared in Comparative Examples. This result confirms that the di-PEG-polypeptide homodimer complex of the invention shows far superior durability in vivo.

### TEST EXAMPLE 6

### Measurement of In Vivo Activity of di-PEG-hGH Homodimer Complex

5 pituitary-removed male Sprague Dawley rats (5-week old, SLC, USA) were employed for each group in a body weight gaining test to measure the in vivo activities of di-PEG-hGH homodimer complex and mono-PEG-hGH. A solvent control, wild-type hGH, mono-PEG-hGH and di-PEG-hGH homodimer complex were subcutaneously injected into the rat's back of the shoulder using a 26G syringe (1 ml, Korea Vaccine Co., Ltd.) according to the administration schedule and dose described in Table 5. Rats' weights were measured before the injection and 16 hours after the injection. Rats were sacrificed with ether 24 hours after the final injection, and the presence of pituitary gland was examined with the naked eye to exclude the rats having observable residual pituitary gland from the result.

TABLE 5
Condition for in vivo activity test of hGH in animal models

| | | Total amount of | Administration |
|---|---|---|---|
| Group | Drug | administration | schedule |
| 1 | Solvent control | PBS (0.5 ml) | Once/day, |
| | | | Daily administration |
| | | | for 6 days |
| 2 | Wild-type hGH | 60 mIU | Once/day, |
| | | (30 μg/time) | Daily administration |
| | | | for 6 days |
| 3 | Mono-PEG-hGH | 360 mIU | Once/6 days, |
| | | (180 μg/time) | Once administration |
| 4 | Di-PEG-hGH | 360 mIU | Once/6 days, |
| | homodimer | (180 μg/time) | Once administration |
| | complex | | |

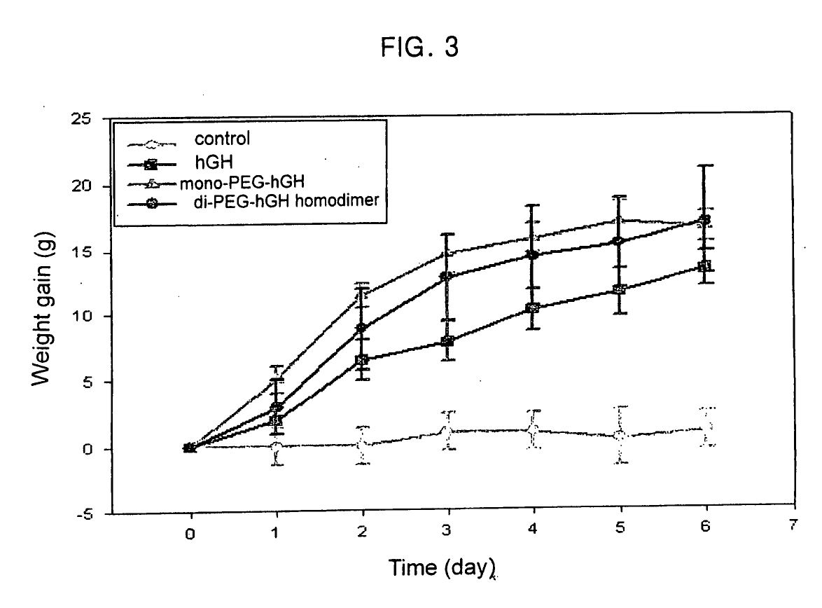

The change in the weight after the administration of each sample was shown in FIG. 3. Since the wild-type hGH used as a standard (control) must be administered everyday to maintain its in vivo activity, it was administered once a day for 6 days, and accordingly, rats of Group 2 gained weight during the administration. Rats of Group 3 administered with the mono-PEG-hGH once/6 days gained weight continuously till 3 days after the administration, and the rate of increase slowed down thereafter, and then, decreased 5 days after the administration. Meanwhile, rats of Group 4 administered with the di-PEG-hGH homodimer complex once/6 days gained weight more slowly than those of Group 3, but the aspect of the rate of increase was very similar to that of Group 2. Further, the rate of increase was on the increase even at day 5 after the administration. Therefore, the di-PEG-hGH homodimer complex of the present invention has a prolonged half-life, while maintaining the activity of the physiologically active polypeptide.

While the invention has been described with respect to the above specific embodiments, it should be recognized that various modifications and changes may be made to the invention by those skilled in the art which also fall within the scope of the invention as defined by the appended claims.

What is claimed is:

1. A PEG-polypeptide homodimer complex comprising a PEG linker and two molecules of a physiologically active polypeptide, wherein the two molecules of the physiologically active polypeptide are connected via the PEG linker, and each of the two molecules of the physiologically active polypeptide is modified with one molecule of PEG.

2. The complex of claim 1, wherein each amino terminal of the two molecules of the physiologically active polypeptide is connected via the PEG linker.

3. The complex of claim 1, wherein the amino group of a lysine residue of the physiologically active polypeptide is modified with said one molecule of PEG.

4. The complex of claim 1, wherein the physiologically active polypeptide is selected from the group consisting of human growth hormone, interferon, granulocyte colony stimulating factor, granulocyte colony stimulating factor derivative having an amino acid sequence wherein cysteine at position 17 is replaced with serine, erythropoietin, insulin, interleukin, granulocyte macrophage colony stimulating factor, and tumor necrosis factor receptor.

5. The complex of claim 1, wherein the PEG linker has two aldehyde or propionic aldehyde groups at both ends.

6. The complex of claim 1, wherein the molecular weight of the PEG linker ranges from 1 to 100 kDa.

7. The complex of claim 6, wherein the molecular weight of the PEG linker ranges from 2 to 20 kDa.

8. The complex of claim 1, wherein said PEG for modifying the physiologically active polypeptide has at one end a reactive group selected from the group consisting of succinimidyl propionate, succinimidyl carboxymethyl, succinimidyl carbonate and maleimide.

9. The complex of claim 1, wherein said PEG for modifying the physiologically active polypeptide is linear or branched.

10. The complex of claim 1, wherein the molecular weight of said PEG for modifying the physiologically active polypeptide ranges from 1 to 100 kDa.

11. The complex of claim 10, wherein the molecular weight of said PEG for modifying the physiologically active polypeptide ranges from 20 to 40 kDa.

12. A method for preparing the PEG-polypeptide homodimer complex of claim 1, which comprises the steps of:
(a) preparing a homodimer by connecting two molecules of a physiologically active polypeptide via a PEG linker; and
(b) modifying each of the two molecules of the physiologically active polypeptide of the homodimer with one molecule of PEG.
